(19) United States
(12) Patent Application Publication
(10) Pub. No.: US 2024/0415406 A1
(43) Pub. Date: Dec. 19, 2024

(54) WIRELESS FREQUENCY-DIVISION MULTIPLEXED 3D MAGNETIC LOCALIZATION FOR LOW POWER SUB-MM PRECISION CAPSULE ENDOSCOPY

(71) Applicant: University of Southern California, Los Angeles, CA (US)

(72) Inventors: Constantine Sideris, Pasadena, CA (US); Michella Rustom, Los Angeles, CA (US)

(21) Appl. No.: 18/719,017

(22) PCT Filed: Oct. 13, 2022
(86) PCT No.: PCT/US2022/046588

§ 371 Date: Jun. 12, 2024

Related U.S. Application Data
(60) Provisional application No. 63/288,774, filed on Dec. 13, 2021.

(51) Int. Cl. A61B 5/06; A61B 1/04; A61B 5/00

(52) U.S. Cl. CPC A61B 5/062; A61B 5/7264; A61B 1/041

(57) ABSTRACT

Disclosed herein are systems and methods related to a capsule endoscopy, where a patient can swallow an ingestible capsule that records images of digestive tract, and a new in-body positioning system can precisely localize the capsule's position. Implementations include a new frequency-division multiplexing-based magnetic localization (FDMML) approach which leverages a higher frequency carrier in the low MHz range. The approach significantly reduces the reference excitation coil sizes and decreases the required excitation current by three orders of magnitude compared to prior work, making it practical for wearable systems. A fully integrated wireless receiver prototype is implemented in 180 nm bulk CMOS and packaged in an ingestible pill form factor. The new scheme achieves the best experimentally demonstrated tracking accuracy in both 2D and 3D localization experiments, achieving a sub-mm mean absolute position error and consuming only 247 pW while running at 100% duty cycle.

This application claims priority to U.S. Provisional Application No. 63/288,774 filed Dec. 13, 2021, which application is hereby incorporated by reference for all that it discloses.

### STATEMENT REGARDING FEDERALLY SPONSORED RESEARCH OR DEVELOPMENT

This invention was made with government support under grant no. N000142114005 awarded by the Office of Naval Research. The government has certain rights in the invention.

### BACKGROUND

The present invention relates generally to the field of capsule endoscopy.

### SUMMARY

Aspects of the present disclosure are directed to a localization scheme for tracking the position of an ingestible pill. The pill may be ingested in order to monitor or diagnose digestive tract issues. Tracking the position of the pill allows for data collected by one or more sensors of the pill to be correlated with a location of the pill as it passes through a digestive tract. A combination of sensor data and location data may assist in monitoring and diagnosing digestive tract issues more accurately than sensor data alone. More accurate position tracking may enable more accurate monitoring and diagnoses.

The systems and methods disclosed herein are related to an ingestible capsule for performing endoscopy. In some implementations, a patient can swallow an ingestible capsule which records images of the digestive tract as it is digested via peristalsis. A new in-body positioning system can precisely localize the capsule's position based on frequency-division multiplexing-based magnetic localization (FDMML) approach. In one aspect, all external magnetic beacons (MBs) are concurrently excited, each having a different small (e.g., 1 Hz-1 MHz range) offset frequency from the carrier (e.g., 1-100 MHz range). The FDMML pill receiver amplifies the voltage picked up by a resonant RX coil from the fields produced by all the MBs, mixes it down to baseband, filters and amplifies it further, and digitizes the data. The digitized data can either be processed directly on chip or transmitted wirelessly for off-chip processing. A suitable inversion algorithm such as one based on an Artificial Neural Network (ANN) can be used to estimate the 3D position of the pill to better than 1 mm precision from the measured field magnitudes from each reference magnetic beacon. The new FDMML scheme achieves sub-mm 3D localization accuracy, real-time 3D tracking, and a 1000× reduction in required excitation coil power, making it suitable for wearable systems.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification. Aspects can be combined and it will be readily appreciated that features described in the context of one aspect of the invention can be combined with other aspects. Aspects can be implemented in any convenient form. As used in the specification and in the claims, the singular form of ‘a’, ‘an’, and ‘the’ include plural referents unless the context clearly dictates otherwise.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings are not intended to be drawn to scale. Like reference numbers and designations in the various drawings indicate like elements. For purposes of clarity, not every component may be labeled in every drawing. In the drawings:

FIG. 1 depicts an example system for performing capsule endoscopy with an ingestible capsule and external magnetic beacons, according to one or more implementations;

FIG. 2 depicts an example environment for performing capsule endoscopy, according to one or more implementations;

FIG. 3A depicts a first view of an ingestible capsule, the tracker sensor, featuring the FDMML scheme, according to one or more implementations.

FIG. 3B depicts a second view of the ingestible capsule of FIG. 3A.

FIG. 4 depicts a low-power complementary metal oxide semiconductor (CMOS) FDMML transceiver chip of the ingestible capsule, according to one or more implementations;

FIG. 5 depicts an example schematic diagram of a circuit board of the ingestible capsule, according to one or more implementations.

FIG. 6 depicts an example schematic diagram of a core operational transconductance amplifier (OTA) of a radio frequency low noise amplifier (RFLNA), a Rauch filter, and a baseband OTA (BBOTA) of a receiver chip of an ingestible capsule, according to one or more implementations;

FIG. 7 depicts an example schematic diagram of a mixer of a receiver chip of an ingestible capsule, according to one or more implementations;

FIG. 8 depicts an example schematic diagram of a binary frequency shift keying (BFSK) voltage-controlled oscillator (VCO) of a receiver chip of an ingestible capsule, according to one or more implementations;

FIG. 9 depicts a schematic diagram of a successive approximation register (SAR) analog-digital converter (ADC) of a receiver chip of an ingestible capsule, according to one or more implementations;

FIG. 10 depicts an example schematic diagram of a magnetic beacon board, according to one or more implementations;

FIG. 11 depicts an example neural network implemented for localization of the ingestible capsule, according to one or more implementations;

FIG. 12 depicts an example flow chart illustrating operations for tracking an ingestible device.

FIG. 13 depicts an example flow chart illustrating operations for tracking an ingestible device.

FIG. 14 depicts a flow chart of a method for wireless frequency-division multiplexed 3D magnetic localization for low power sub-mm precision capsule endoscopy, according to one or more implementations.

FIG. 15 is a table illustrating example power usage of the circuit board of the ingestible capsule while running at 100% duty cycle.

### DETAILED DESCRIPTION

Below are detailed descriptions of various concepts related to, and implementations of, techniques, approaches, methods, apparatuses, and systems for generating dynamic content sets based on real-time feedback. The various concepts introduced above and discussed in greater detail below may be implemented in any of numerous ways, as the described concepts are not limited to any particular manner of implementation. Examples of specific implementations and applications are provided primarily for illustrative purposes.

Over 15 million colonoscopies are performed yearly in the US. Capsule endoscopy has emerged as a noninvasive alternative, which relieves patients of the discomfort of a colonoscopy. A patient swallows an ingestible capsule which records images of the digestive tract as it is digested via peristalsis. Some products can capture and transmit images in real-time; however, they are unable to precisely localize the capsule's position as it is digested. Localization is crucial for diagnosis as it can tag images with 3D spatial information and inform physicians of the location of abnormalities. Although many modalities for localization have been explored (e.g., optical, radio-frequency, microwave imaging, static and alternating magnetic fields), magnetic field approaches have emerged as the most precise. This is because the body exhibits almost no magnetic effects (μᵣ≈1) and is transparent to magnetic fields.

Apart from being precise, the localization scheme should support a large spatial range to track the position inside the human body. It should be fully integrated and consume very low power so that it can co-exist with the rest of the capsule imaging electronics and run off a low-capacity battery. Some magnetic localization schemes use either static magnetic fields or very low-frequency (10-100 Hz) alternating fields and determine the relative capsule position with respect to the external magnetic beacon (MB) references by sensing magnetic field magnitudes and solving an inverse problem. These approaches have very high-power requirements for the beacon coils and may suffer from poor accuracy due to the low sensitivity of DC Hall effect sensors compared to AC inductive resonant coils, interfering DC fields (e.g., the Earth's magnetic field), and the high flicker noise of active readout circuitry at low frequencies.

Embodiments discussed herein represent a new frequency-division multiplexing-based magnetic localization (FDMML) scheme which leverages a frequency carrier in the low MHz range (e.g., 1-100 MHz). Multiple magnetic beacons may concurrently generate magnetic fields with a baseline frequency 1-100 MHz. Each beacon may utilize a different offset frequency in the Hz to kHz range, allowing for an ingestible pill to differentiate between magnetic fields generated by different beacons. The pill may include a reception coil for measuring the magnetic fields from the beacons. The pill may include a transmission coil for transmitting localization data to the beacons. In some arrangements, the localization data may include magnetic field measurements for each magnetic field generated by the beacons. A neural network may determine location data of the pill relative to the beacons. In other arrangements, the localization data may include the location data of the pill relative to the beacons, as calculated by the pill based on the magnetic field measurements.

This new FDMML scheme can provide at least the technical improvement of reducing magnetic beacon coil sizes and increasing the voltage picked up at the pill for the same beacon power transmitted. Lower beacon power may be used than would be necessary for generating magnetic fields at a lower frequency. In addition, using frequencies above a flicker noise corner of a CMOS device of the pill enables high-sensitivity reception via low-noise amplification before down-conversion. Furthermore, using a MHz-range frequency allows the magnetic beacons to use the offset frequencies, allowing for the beacons to generate magnetic fields concurrently. Concurrent generation from each magnetic beacon shortens an amount of time required to read the magnetic fields from different beacons relative to sequential generation, allowing for faster, more accurate, and more granular localization data. Furthermore, concurrent generation from each magnetic beacon allows for each beacon to generate a magnetic field with a sustained current, using much less power relative to sequential generation which requires repeatedly pulsing large currents into each coil.

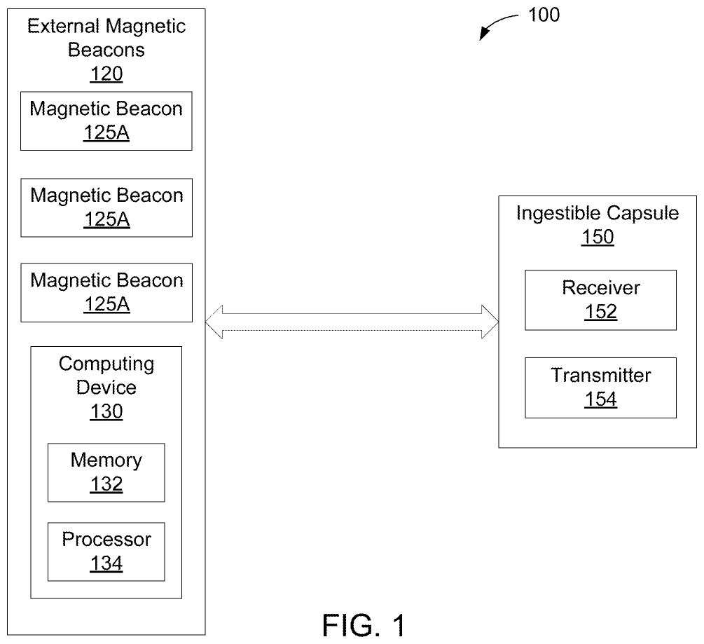

FIG. 1 depicts an example system 100 for performing capsule endoscopy with an ingestible capsule 150 and external magnetic beacons 120, according to one or more implementations. In one aspect, the ingestible capsule 150 can be ingested by a patient. The ingestible capsule 150 may capture images of digestive tract of the patient. The external beacon circuitry 120 may generate magnetic fields at different small offset frequencies with respect to a base carrier, measured by the ingestible capsule 150. The external beacon circuitry 120 may include one or more magnetic beacons 125A-125C and a computing device 130. Although in FIG. 1, the external excitation circuitry 120 is shown as including three magnetic beacons 125A-125C, the external excitation circuitry 120 may include any number of magnetic beacons 125. In some arrangements, the ingestible capsule 150 may be an ingestible device. The ingestible device may be in capsule form or in another form. The ingestible device may be in any form capable of being swallowed by the patient. The ingestible capsule 150 may include a receiver 152 and a transmitter 154.

A computing device 130 may configure operations of the magnetic beacons 125. In some arrangements, the computing device 130 may be a controller of the magnetic beacons 125. The computing device 130 may receive measurement data from the capsule transceiver 125A-125C, and determine a location of the ingestible capsule 150. In one example, the computing device 130 implements a neural network model to determine the location of the ingestible capsule 150. In one aspect, various measurements from multiple magnetic beacons 125A-125C can enable accurate tracking of the ingestible capsule 150. The computing device 130 may include a memory 132 and a processor 134. The memory 132 may be a non-transitory, computer-readable medium including instructions which, when executed by the processor 134, cause the processor 134 to perform various operations as discussed herein.

In one aspect, the magnetic beacon 125 can operate in a power and time efficient manner. In one example, different magnetic beacon coils can be pulsed on and off sequentially, but such an approach may be inefficient in terms of time and power consumption. In one aspect, different magnetic beacons 125 may generate magnetic fields with different small offset frequencies (e.g., Hz to MHz range) with respect to a carrier frequency, such that different magnetic beacons can be concurrently enabled. Aside from reducing readout time considerably, which is important for tracking a moving capsule, concurrent readout using high-Q resonant coils with a sustained current offers significant power savings over repeatedly pulsing large currents into each coil. FDMML can reduce the excitation coil current by up to three orders of magnitude compared to other approaches.

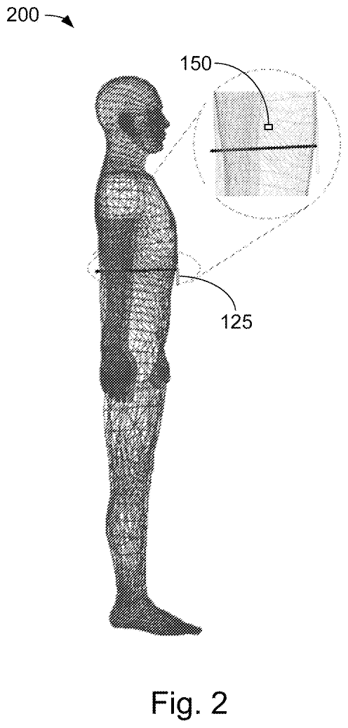

FIG. 2 depicts an example environment 200 for performing capsule endoscopy, according to one or more implementations. The ingestible capsule 150 and a magnetic beacon 125 of FIG. 1 may be used to perform capsule endoscopy while tracking a position of the ingestible capsule 150. In some arrangements, the magnetic beacon 125 may be worn by a patient. In some arrangements, multiple magnetic beacons may be worn by the patient.

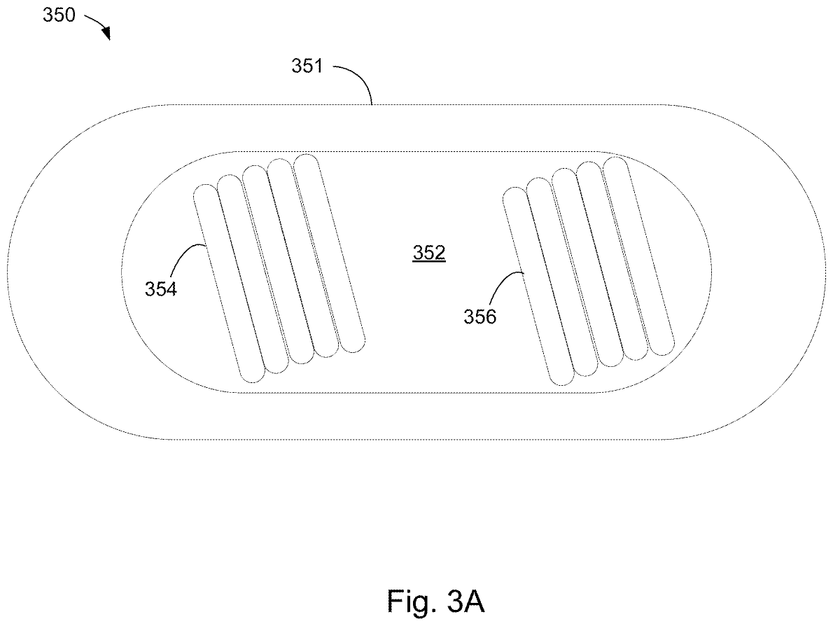

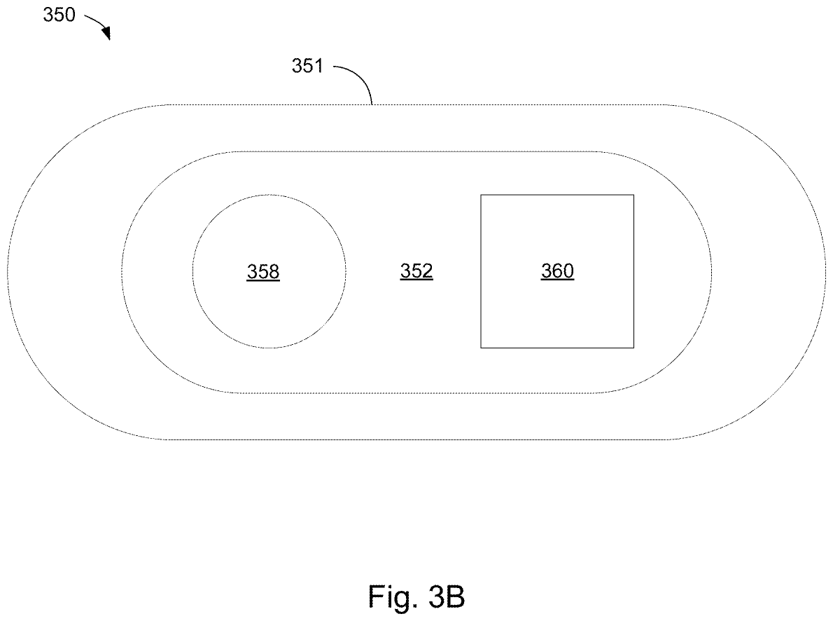

FIG. 3A depicts a first view of an ingestible capsule 350 that can be tracked using the FDMML approach, according to one or more implementations. In some arrangements, the ingestible capsule 350 may be the ingestible capsule 150 of FIG. 1. As shown in FIG. 3A, the ingestible capsule 350 can be implemented in a small form factor by utilizing FDMML over a frequency band (e.g., 1-100 MHz), such that the ingestible capsule 350 can be swallowed by the patient for endoscopy exam. The ingestible capsule 350 may include an outer shell 351. In some arrangements, the outer shell 351 may be capsule-shaped. In other arrangements, the outer shell 351 may be another shape. The ingestible capsule 350 may include a circuit board 352. The circuit board may include a receive coil 354 and a transmit coil 356. The receive coil 354 may be configured to resonate in response to magnetic fields generated by magnetic beacons and sense corresponding voltages. The circuit board 352 may be configured to filter the voltages, amplify the voltages, digitize the voltages, generate measurement data containing the spatial information of the pill, and drive the transmit coil 356 to transmit information. In some arrangements, the transmit coil 356 transmits the measurement data generated by the circuit board 352 based on the detected magnetic fields. In other arrangements, the circuit board 354 transmits location data of the ingestible capsule 350 based on the measurement data.

FIG. 3B depicts a second view of the ingestible capsule 350 of FIG. 3A. The ingestible capsule 350 may include a battery 358 and a chip 360. The battery 358 and the chip 360 may be on a side of the circuit board 352 opposite the receive coil 354 and the transmit coil 356. The battery 358 may provide power to the chip 360 and the transmit coil. The chip 360 may be configured to receive the voltages sensed by the receive coil 354 and filter the voltages, amplify the voltages, digitize the voltages, and generate measurement data, as discussed herein. In some arrangements, the chip 360 may be configured to drive the transmit coil 356 to transmit information.

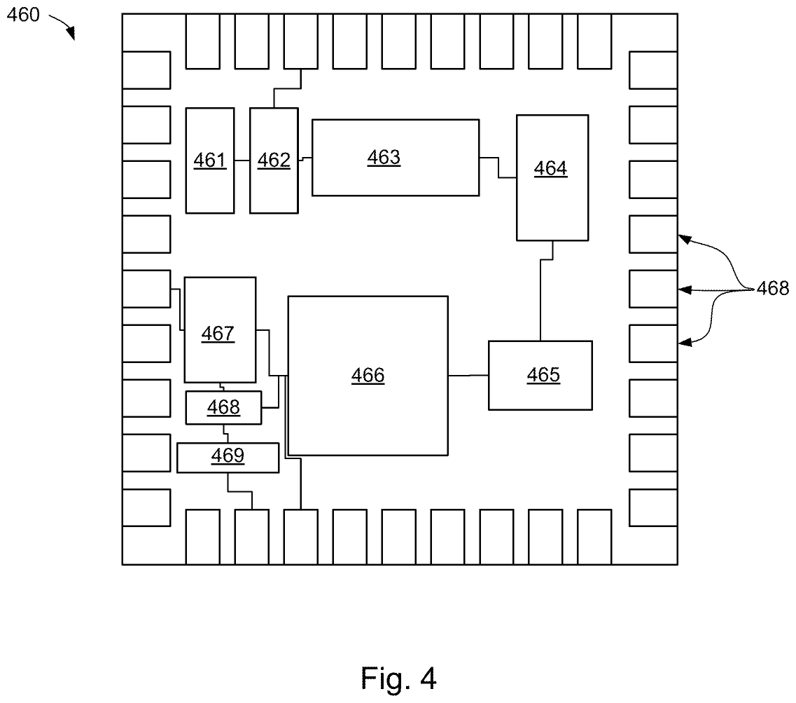

FIG. 4 depicts a low-power CMOS FDMML receiver chip 460 of the ingestible capsule, according to one or more implementations. In some arrangements, the receiver chip 460 may be the chip 360 of FIG. 3B. The receiver chip 460 may include an RFLNA 461, a mixer 462, a Rauch biquadratic cell 463, a first BBOTA 464, a second BBOTA 465, an SAR ADC 466, a BFSK VCO 467, a pseudorandom binary sequence (PRBS) 468, a digital control 469, and a plurality of contacts 468.

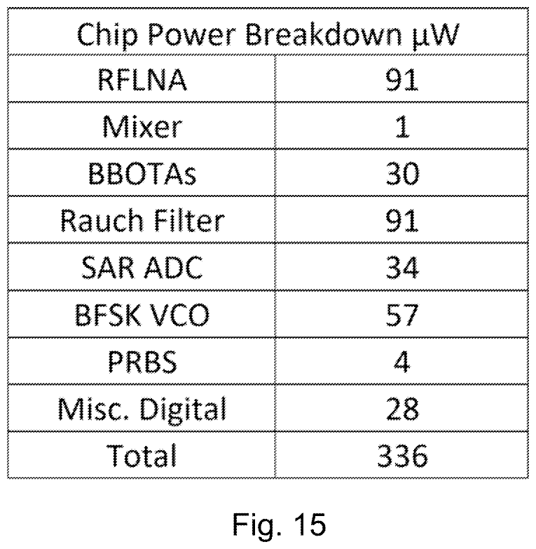

The ingestible capsule may be implemented in a bulk 180 nm CMOS process or any other node. The receiver chip 460 may amplify a voltage picked up by a resonant receive coil from the fields produced by all the MBs, mix it down to baseband, filter and amplify it further, and digitize the data using the SAR ADC 466. The data may be transmitted wirelessly via BFSK by directly modulating a power oscillator such as the BFSK VCO 467 using a transmit coil as a resonator. Since field perturbations do not matter on the transmit side, a higher 57 MHz transmit carrier frequency can be chosen to avoid crosstalk with the receive coil and maximize wireless link range. The only off-chip components may be the receive and transmit coils (both 160 nH), their corresponding resonating capacitors, and an ultra-low power 2.048 MHz MEMS oscillator for driving the mixer linear oscillator (LO), allowing the whole system to fit in the ingestible capsule 350. The RFLNA 461 high-input impedance stage may be a differential two stage current mirror OTA with capacitive feedback and may provide a 32 dB gain with 9 MHz 3 dB bandwidth and 26 nV/VHz simulated input referred noise. The RFLNA 461 may be followed by the mixer 462. In some arrangements, the mixer may be a double balanced high-linearity, low-noise passive mixer with an active single-ended to differential conversion circuit for the LO input. The Rauch filter 463 may low-pass filter the mixer output and drive the first and second BBOTAs 464, 465. In some arrangements, the Rauch filter 463 may be a unity gain Rauch filter with programmable switched capacitors to compensate process variation. In some arrangements, the first and second BBOTAs may be cascaded capacitive-feedback baseband OTAs (BBOTA), each with 4-bit programmable gain control. The filter bandwidth may be adjustable from 40 to 150 kHz (nominal 98 kHz) with 2nd order Butterworth response, and each of the first and second BBOTAs 464, 465 may have configurable gain from 6 to 18 dB and 200 kHz bandwidth. The total voltage gain, considering 3.9 dB mixer conversion loss, may be set from 40 to 64 dB allowing for a wide input dynamic range. The signal may be sampled using a bootstrapped switch sampler and digitized using the SAR ADC 466. In some arrangements, the SAR ADC 466 may be a 10-bit monotonically switched SAR ADC, operated at a 51.2 KSPS sampling rate by dividing down the reference clock using digital flip-flop dividers. The digitized bits from the SAR ADC 466 may be shifted out to drive the BFSK VCO 467. In some arrangements, the BFSK VCO 467 may process the digitized bits at 1.024 Mbps. In some arrangements, the BFSK VCO 467 may be part of a BFSK transmitter, which includes the BFSK VCO 467 which may be differential cross-coupled with switched capacitors for modulating the oscillation frequency. Either one or both capacitor cells (each contributing 3.5 pF to the tank) of the BFSK VCO 467 may be switched to select the frequency modulation depth. A BFSK VCO 467 current may be digitally adjustable from 32 to 630 μA. The PRBS 468 may characterize the wireless communication. In some arrangements, the PRBS 468 may be a 7-bit PRBS. In some arrangements, during an ADC conversion phase, 10 PRBS bits may be transmitted serving as a frame identifier. During a sampling phase, the 10 bits from the previous conversion cycle may be shifted out and transmitted. BFSK may be used instead of amplitude shift keying (ASK) since it is more robust to noise and more energy efficient when using an oscillator-based transmit coil as it does not suffer from a Q vs. bandwidth tradeoff. In some arrangements, total chip power consumption at the lowest VCO setting may be 336 μW off a 1.8V supply while running at 100% duty cycle, which can be reduced using power gating due to slow movement inside the body. By implementing the RFLNA 461, the passive mixer 462, the Rauch filter 463, the first BBOTA 464, the second BBOTA 465, the BFSK VCO 467, and SAR ADC 466, the ingestible capsule can operate with low power consumption as shown in FIG. 15.

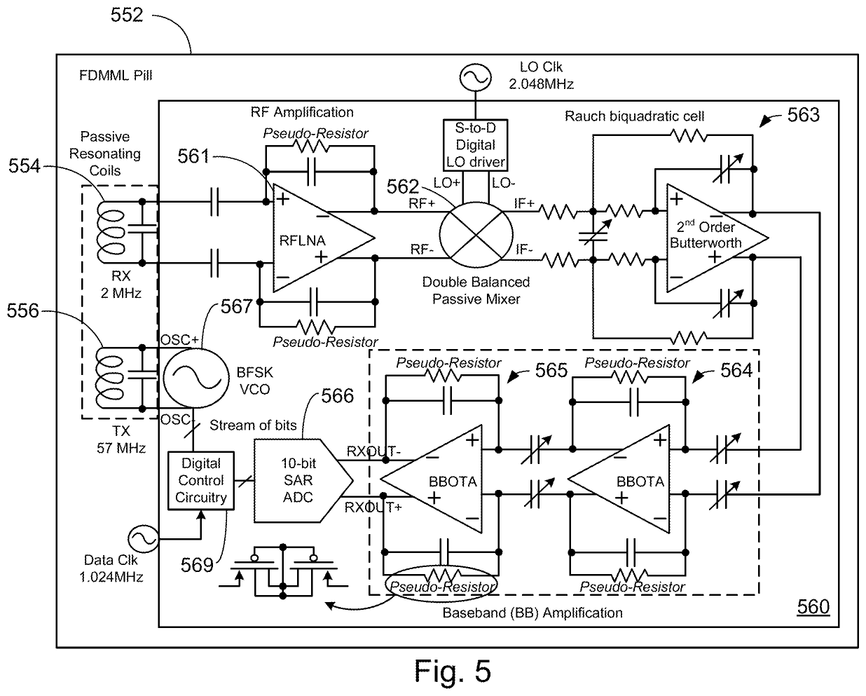

FIG. 5 depicts an example schematic diagram of a circuit board 552 of an ingestible capsule, according to one or more implementations. In some arrangements, the circuit board 552 may be the circuit board 352 of FIGS. 3A and 3B. The circuit board 552 may include a receive coil 554 and a transmit coil 556. In some arrangements, the receive coil 554 and the transmit coil 556 may be the receive coil 354 and transmit coil 356, respectively, of FIG. 3A. The circuit board 552 may include a chip 560. In some arrangements, the chip 560 may be the chip 460 of FIG. 4. The chip 560 may include an RFLNA 561, a mixer 562, a Rauch cell 563, a first and second BBOTA 564, 565, a SAR ADC 566, a BFSK VCO 567, and a digital control circuitry 569.

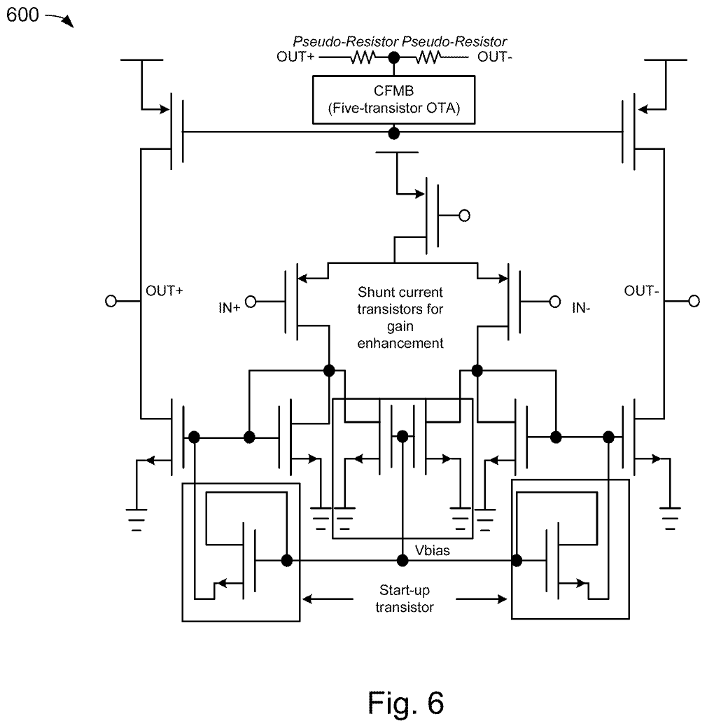

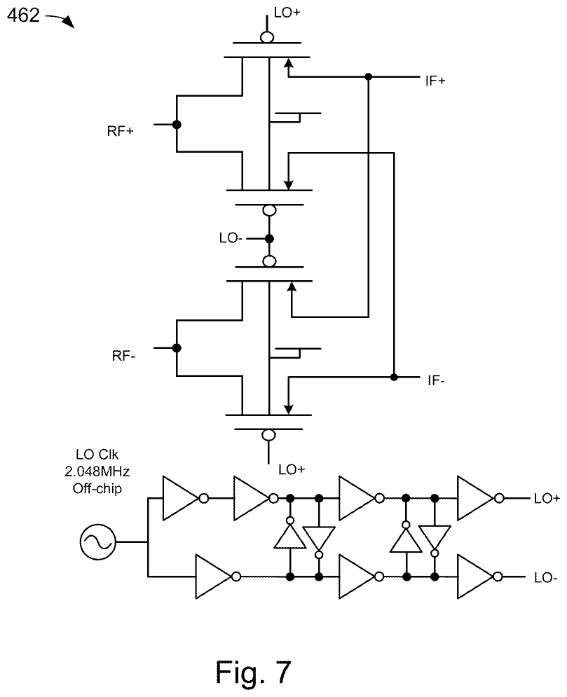

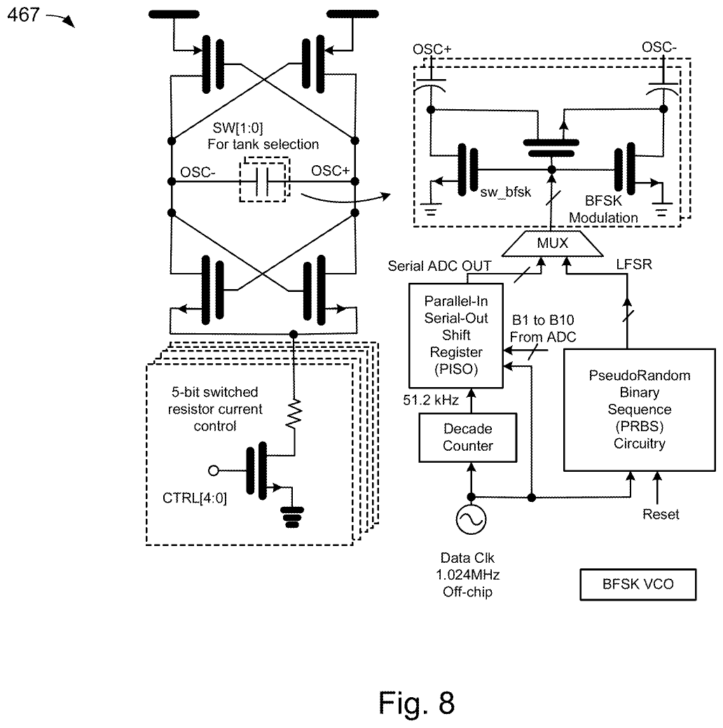

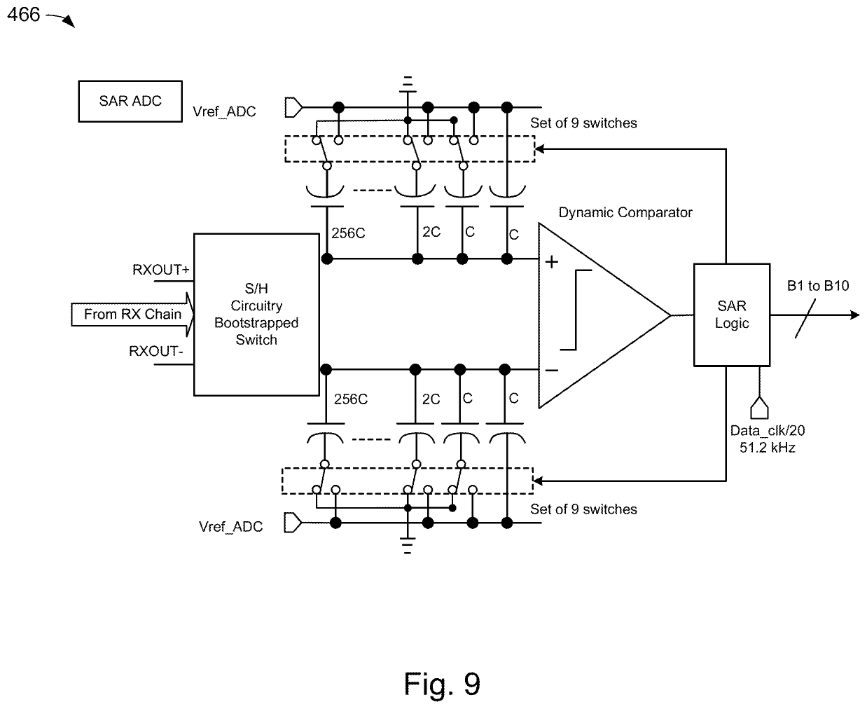

An example implementation of the core OTA of the RFLNA 461, Rauch filter 463, and first and second BBOTAs 464, 465 of the receiver chip 460 of FIG. 4 is shown in FIG. 6. An example implementation of the mixer 462 of FIG. 4 is shown in FIG. 7. An example implementation of the BFSK VCO 467 of FIG. 4 is shown in FIG. 8. An example implementation of the SAR ADC 466 of FIG. 4 is shown in FIG. 9.

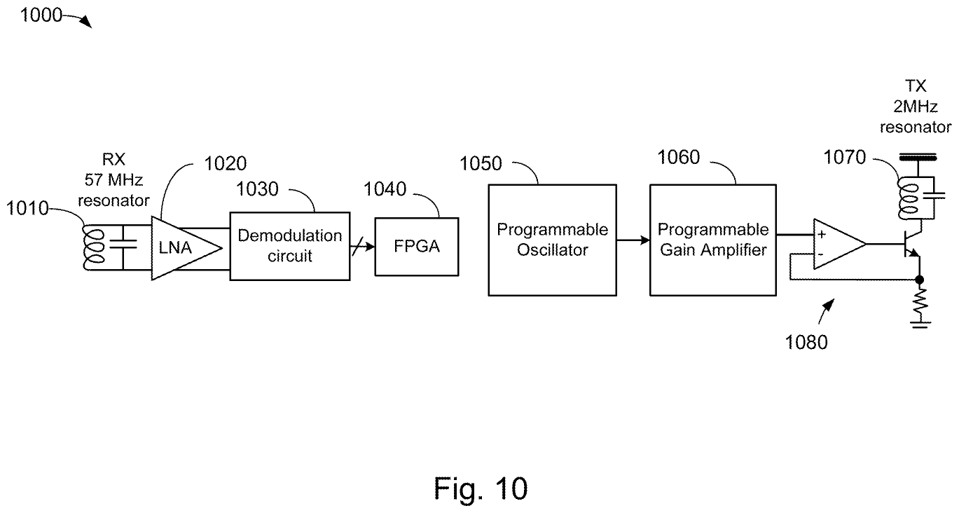

FIG. 10 depicts a schematic diagram of a magnetic beacon 1000, according to one or more implementations. The magnetic beacon 100 may include a receive resonator 1010, a low noise amplifier (LNA) 1020, a demodulation circuit 1030, a field-programmable gate array (FPGA) 1040, a programmable oscillator 1050, a programmable gain amplifier 1060, a transmit resonator 1070, and a feedback circuit. The receive resonator 1010 and the transmit resonator 1070 may be on-board planar, concentric coils. In some arrangements, the transmit resonator 1070 may be a 2 MHz coil and the receive resonator 1010 may be a 57 MHz coil. The feedback circuit 1080 may enable current control through the transmit coil 1070 driven by a programmable reference VCO. In some arrangements, the receive resonator 1010 may be an FSK receiver. The receive resonator 1010 may receive digitized data from an ingestible capsule or ingestible device and send the digitized received data to the FPGA 1040 for computer (e.g., computing device 130) readout via USB.

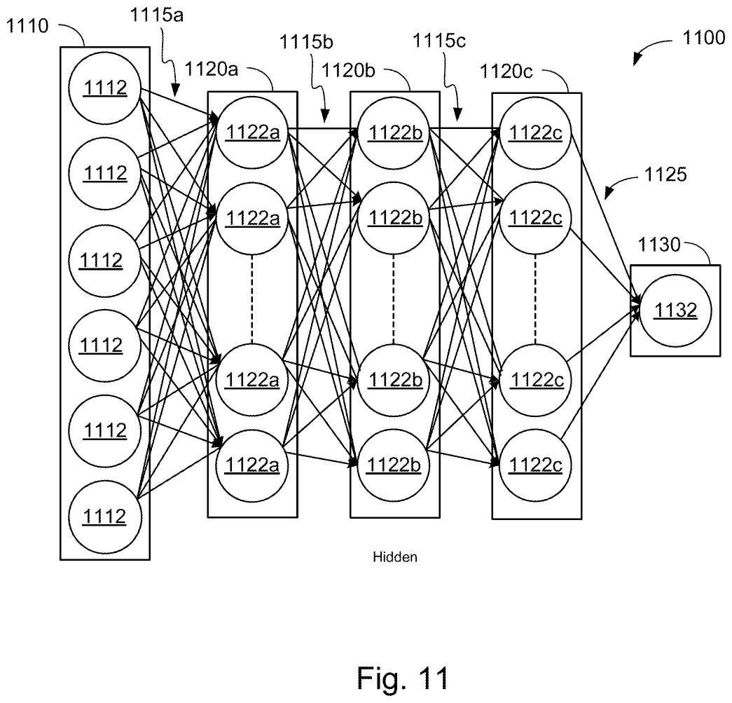

FIG. 11 depicts an example neural network 1100 implemented for localization of the ingestible capsule, according to one or more implementations. In some arrangements, the computing device 130 of FIG. 1 may implement the neural network 1100. The neural network 1100 may be trained such that the neural network receives measurement generated by an ingestible device from one or more magnetic beacons 125, and generates data indicating a location of the ingestible device in response to the measurement data. The neural network 1100 may include an input layer 1110, an output layer 1130, a first hidden layer 1120a, a second hidden layer 1120b, and a third hidden layer 1120c. Although the neural network 1100 is shown having three hidden layers 1120a, 1120b, and 1120c, the neural network may include any number of hidden layers 1120. The neural network 1100 may be a deep neural network. The input layer 1110 may include as input magnetic field magnitudes corresponding to different magnetic beacons. The input layer 1110 may receive the input from the magnetic beacons and/or a magnetic beacon receiver. The input layer 1110 may include a plurality of input layer nodes 1112. Each node of the plurality of input layer nodes 1112 may perform transform operations on the received input.

The first hidden layer 1120a may include a plurality of first hidden layer nodes 1122a. The plurality of first hidden layer nodes 1122a may perform transform operations on input received from the input layer 1110. The plurality of input layer nodes 1112 may be connected to the plurality of first hidden layer nodes 1122a by a plurality of first edges 1115a. In the illustrated example, a plurality of edges connects each layer, but in some embodiments, a plurality of edges is not required. The plurality of first edges 1115a may connect any number of the plurality of input layer nodes 1112 to any number of the plurality of first hidden layer nodes 1122a.

The second hidden layer 1120b may include a plurality of second hidden layer nodes 1122b. The plurality of second hidden layer nodes 1122b may perform transform operations on input received from the first hidden layer 1120a. The plurality of first hidden layer nodes 1122a may be connected to the plurality of second hidden layer nodes 1122b by a plurality of second edges 1115b. The plurality of second edges 1115b may connect any number of the plurality of first hidden layer nodes 1122a to any number of the plurality of second hidden layer nodes 1122b.

The third hidden layer 1120c may include a plurality of third hidden layer nodes 1122c. The plurality of third hidden layer nodes 1122c may perform transform operations on input received from the second hidden layer 1120b. The plurality of second hidden layer nodes 1122b may be connected to the plurality of third hidden layer nodes 1122c by a plurality of third edges 1115c. The plurality of third edges 1115c may connect any number of the plurality of second hidden layer nodes 1122b to any number of the plurality of third hidden layer nodes 1122c.

The output layer 1130 may include an output node 1132. The output node 1132 may perform transform operations on input received from the third hidden layer 1120c. The plurality of third hidden layer nodes 1122c may be connected to the output node 1132 by a plurality of fourth edges 1125. The plurality of fourth edges 1125 may connect any number of the plurality of third hidden layer nodes 1122c to the output node 1132. The output node 1132 may output a position of the ingestible capsule. In some embodiments, the output node 1132 may output the position of the ingestible capsule in Cartesian coordinates. In some embodiments, the output node 1132 may output the position of the ingestible capsule along an axis.

In an example, six magnetic beacons concurrently generate magnetic fields with different offset frequencies. An ingestible capsule measures a magnitude of each of the six magnetic fields and transmits the six magnetic field magnitudes to a magnetic beacon receiver. The six magnetic field magnitudes are used as input in the input layer 1110. Each layer of the neural network 1100 performs calculations on input from a preceding layer until the output layer 1130 outputs the position of the ingestible capsule.

Above a certain frequency, the body may perturb the fields appreciably due to dielectric effects, resulting in position tracking errors. In order to determine the maximum acceptable frequency, we performed electromagnetic simulations using a human body model with varying excitation coil frequencies and plotted the fields measured inside the body vs. in vacuum. The two cases are almost indistinguishable from each other at 1 and 10 MHz and only start to visibly deviate above 30 MHz. A 2.048 MHz center frequency can be selected to maximize inductive link sensitivity and minimize the effects of the body on the fields.

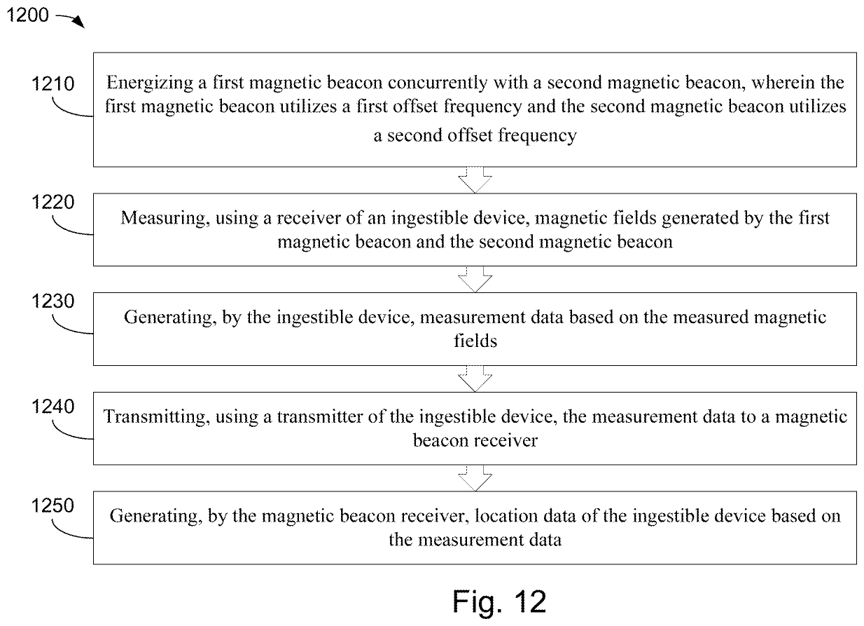

FIG. 12 depicts an example flow chart 1200 illustrating operations for tracking an ingestible device. The operations may include more, fewer, or different steps than illustrated here. The steps may be performed in the order shown, in a different order, or concurrently. The operations may include energizing 1210 a first magnetic beacon concurrently with a second magnetic beacon, wherein the first magnetic beacon utilizes a first offset frequency and the second magnetic beacon utilizes a second offset frequency. The first and second magnetic beacons may use a common carrier frequency in the range of 1-100 MHz. The first and second offset frequencies may be in a range of 1 Hz-1 MHz. The offset frequencies may serve to differentiate magnetic fields generated by the first and second magnetic beacons. The operations may include measuring 1220, using a receiver of an ingestible device, magnetic fields generated by the first magnetic beacon and the second magnetic beacon. Measuring the magnetic fields may include measuring voltages generated or picked up by a receiver coil, as discussed herein. The first and second offset frequencies may result in different voltages at different frequencies, allowing the ingestible device to differentiate between the magnetic fields. The operations may include generating 1230, by the ingestible device, measurement data based on the measured magnetic fields. Generating the measurement data may include amplifying, filtering, and digitizing the voltages picked up or generated by the receiver coil, as discussed herein. The operations may include transmitting 1240, using a transmitter of the ingestible device, the measurement data to a magnetic beacon receiver. The transmitter of the ingestible device may be a transmit coil, as discussed herein. In some arrangements, the magnetic beacon receiver may be a component of the magnetic beacon, as discussed herein. In other arrangements, the magnetic beacon receiver may be a receiver associated with the magnetic beacons. The operations may include generating 1250, by the magnetic beacon receiver, location data of the ingestible device based on the measurement data. In some arrangements, generating the location data may include applying a neural network on the measurement data to generate the location data. The neural network may be trained to receive as input magnetic field measurement data and output location data of the ingestible device. In some arrangements, the ingestible device may generate the location data and transmit the location data to the magnetic beacon receiver.

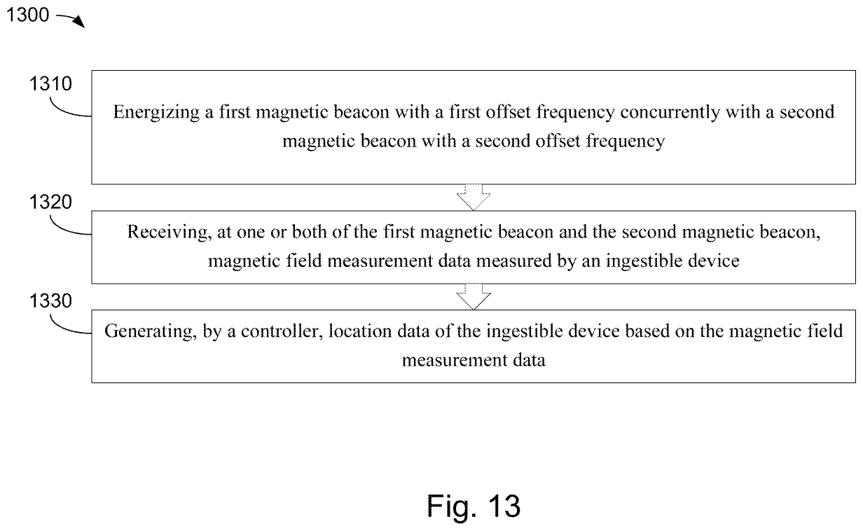

FIG. 13 depicts an example flow chart 1300 illustrating operations for tracking an ingestible device. The operations may include more, fewer, or different steps than illustrated here. The steps may be performed in the order shown, in a different order, or concurrently. The operations may include energizing 1310 a first magnetic beacon with a first offset frequency concurrently with a second magnetic beacon with a second offset frequency. The first and second magnetic beacons may use a common carrier frequency in the range of 1-100 MHz. The first and second offset frequencies may be in a range of 1 Hz-1 MHz. The offset frequencies may serve to differentiate magnetic fields generated by the first and second magnetic beacons. The operations may include receiving 1320, at one or both of the first magnetic beacon and the second magnetic beacon, magnetic field measurement data measured by the ingestible device. The ingestible device may generate the measurement data based on the magnetic fields generated by the first and second magnetic beacons, as discussed herein. The operations may include generating, 1330, by a controller, location data of the ingestible device based on the magnetic field measurement data. The controller may be a controller of the first and second magnetic beacons. In some arrangements, the controller may be a computing device including a memory and a processor. In some arrangements, the controller may utilize a neural network to generate the location data by applying the neural network on the measurement data.

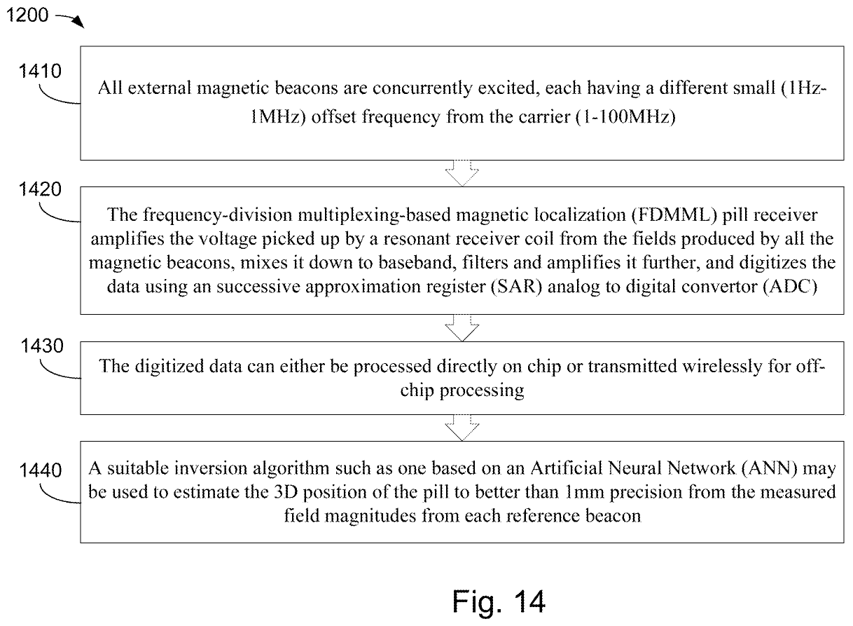

FIG. 14 depicts a flow chart of a method for wireless frequency-division multiplexed 3D magnetic localization for low power sub-mm precision capsule endoscopy, according to one or more implementations. The operations may include more, fewer, or different steps than illustrated here. The steps may be performed in the order shown, in a different order, or concurrently. At 1410, all external magnetic beacons are concurrently excited, each having a different small (1 Hz-1 MHz) offset frequency from the carrier (1-100 MHz). The magnetic beacons may be external to a body of a patient who has ingested an ingestible device. In some arrangements, the ingestible device may be a capsule or pill. At 1420, a frequency-division multiplexing-based magnetic localization (FDMML) pill receiver amplifies a voltage picked up by a resonant receiver coil from the fields produced by all the magnetic beacons, mixes it down to baseband, filters and amplifies it further, and digitizes the data using a successive approximation register (SAR) analog to digital converter (ADC). Mixing the voltage to baseband, filtering the voltage, and amplifying the voltage may allow for different voltages at different frequencies to be detected in order to determine signal strength from each of the magnetic beacons. The measurement data may include a voltage magnitude for each frequency corresponding to each magnetic beacon. At 1430, the digitized data can either be processed directly on chip or transmitted wirelessly for off-chip processing. At 1440, a suitable inversion algorithm such as one based on an Artificial Neural Network (ANN) may be used to estimate the 3D position of the pill to better than 1 mm precision from the measured field magnitudes from each reference beacon.

Existing spatial localization schemes do not provide enough accuracy or precision for 3D localization for capsule endoscopy.

The frequency-division multiplexing-based magnetic localization (FDMML) approach being claimed offers a sub-millimeter 3D tracking resolution and supports a large spatial range for tracking the position of an ingestible pill throughout the digestive tract, by measuring the magnetic fields generated by one or more external magnetic beacons producing magnetic fields in the lower MegaHertz frequency range and determining relative position with respect to the beacons by solving an inverse problem.

Previous magnetic localization schemes, which use either static magnetic fields or very low-frequency (10-100 Hz) alternating fields, have very high-power requirements for the beacon coils.

The FDMML technique leverages a higher frequency carrier in the low MegaHertz range for each external magnetic beacon, thereby reducing the required coil size and boosting the signal strength sensed by a receiver, requiring 1000× less power than static and very low-frequency alternating field localization methods.

Static or very low-frequency (10-100 Hz) alternating field-based localization techniques require exciting each magnetic beacon one at a time in sequence. This results both in very poor energy efficiency due to needing to charge and discharge each coil repeatedly, as well as slow localization times due to requiring to cycle through powering every magnetic beacon coil on and off sequentially per cycle.

Conversely, the FDMML technique enables concurrent read-out from all magnetic beacons by exciting them all at once compared to prior techniques in which the magnetic beacons are pulsed on and off sequentially. All the magnetic beacons utilize the same base carrier frequency in the lower Megahertz range (e.g., 1-100 MHz) and each beacon contains a low-frequency (1 Hz-1 MHz) frequency offset from the base carrier to enable the receiver to distinguish the magnetic field profiles from each magnetic beacon by using frequency-division multiplexing. This reduces the readout time required to localize the receiver considerably, for example a capsule for capsule endoscopy applications, improving dynamic tracking performance significantly for applications where the object to be tracked is moving.

The receiver circuitry required for sensing the fields produced by the FDMML approach and determining its 3D spatial position can be fully integrated, implemented in a standard, silicon CMOS foundry process and packaged in a standard ingestible capsule form factor. A prototype implementation, which includes wireless data transfer of the received data in digital format back to an external recording system, consumes less than 336 μW of total power. The localization circuitry alone (without the digital wireless transmission circuits) consumes only 247 μW of power while running at 100% duty cycle.

Prior localization techniques have large currents pulsed repeatedly and successively into each coil, making the tracking setup time and power inefficient. The higher base carrier frequency of the FDMML approach enables using smaller, high-Quality factor resonant coils with a sustained continuous-wave (CW) current into each coil which offers significant power savings. It reduces the required excitation coil current by up to three orders of magnitude compared to prior work.

The FDMML approach makes the tracking setup compact by miniaturizing both the magnetic beacon coils and the receiver and reducing the total required power down to milliwatt level for the external beacons and microwatt level for the receiver. This enables portable implementation and makes the approach practical for translation to clinical practices and suitable for wearable systems.

Having now described some illustrative implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements and features discussed only in connection with one implementation are not intended to be excluded from a similar role in other implementations or implementations.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act or element may include implementations where the act or element is based at least in part on any information, act, or element.

Any implementation disclosed herein may be combined with any other implementation, and references to “an implementation,” “some implementations,” “an alternate implementation,” “various implementation,” “one implementation” or the like are not necessarily mutually exclusive and are intended to indicate that a particular feature, structure, or characteristic described in connection with the implementation may be included in at least one implementation. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms.

Where technical features in the drawings, detailed description or any claim are followed by reference signs, the reference signs have been included for the sole purpose of increasing the intelligibility of the drawings, detailed description, and claims. Accordingly, neither the reference signs nor their absence have any limiting effect on the scope of any claim elements.

The systems and methods described herein may be embodied in other specific forms without departing from the characteristics thereof. Although the examples provided may be useful for multiwavelet-based operator learning for differential equations, the systems and methods described herein may be applied to other environments. The foregoing implementations are illustrative rather than limiting of the described systems and methods. The scope of the systems and methods described herein may thus be indicated by the appended claims, rather than the foregoing description, and changes that come within the meaning and range of equivalency of the claims are embraced therein.

What is claimed is:

1. A method comprising:
energizing a first magnetic beacon concurrently with a second magnetic beacon, wherein the first magnetic beacon utilizes a first offset frequency and the second magnetic beacon utilizes a second offset frequency;
measuring, using a receiver of an ingestible device, magnetic fields generated by the first magnetic beacon and the second magnetic beacon;
generating, by the ingestible device, measurement data based on the measured magnetic fields;
transmitting, using a transmitter of the ingestible device, the measurement data to a magnetic beacon receiver; and
generating, by the magnetic beacon receiver, location data of the ingestible device based on the measurement data.

2. The method of claim 1, wherein the ingestible device comprises a receiving coil and a transmission coil, wherein the transmission coil uses a frequency greater than 50 MHz.

3. The method of claim 1, wherein the ingestible device comprises a chip including a radio frequency low noise amplifier (RFLNA) configured to amplify measurements of the magnetic fields generated by the first magnetic beacon and the second magnetic beacon.

4. The method of claim 1, wherein generating the location data comprises applying a neural network on the measurement data.

5. The method of claim 1, wherein generating the location data comprises calculating, by the ingestible device, the location data based on the measurement data, and wherein transmitting the measurement data comprises transmitting the location data.

6. The method of claim 1, wherein energizing the first magnetic beacon and the second magnetic beacon comprises energizing the first magnetic beacon and the second magnetic beacon with a sustained current.

7. The method of claim 1, wherein the first magnetic beacon and the second magnetic beacon utilize a carrier frequency in a range of about 1-100 MHz.

8. A system comprising:
a first magnetic beacon configured to generate a first magnetic field with a first offset frequency;
a second magnetic beacon configured to generate a second magnetic field with a second offset frequency; and
a controller comprising a processor and a non-transitory computer-readable medium comprising instructions which, when executed by the processor, cause the processor to:
concurrently energize the first magnetic beacon and the second magnetic beacon;
receive, from the first magnetic beacon and the second magnetic beacon, magnetic field measurement data measured by an ingestible device; and
generate location data of the ingestible device based on the magnetic field measurement data.

9. The system of claim 8, wherein the ingestible device comprises a receiving coil and a transmission coil, wherein the transmission coil uses a frequency greater than 50 MHz.

10. The system of claim 8, wherein the ingestible device comprises a chip including a radio frequency low noise amplifier (RFLNA) configured to amplify measurements of the magnetic fields generated by the first magnetic beacon and the second magnetic beacon.

11. The system of claim 8, wherein generating the location data comprises applying a neural network on the measurement data.

12. The system of claim 8, wherein generating the location data comprises receiving, from the ingestible device, the location data as calculated by the ingestible device.

13. The system of claim 8, wherein energizing the first magnetic beacon and the second magnetic beacon comprises energizing the first magnetic beacon and the second magnetic beacon with a sustained current.

14. The system of claim 8, wherein the first magnetic beacon and the second magnetic beacon utilize a carrier frequency in a range of about 1-100 MHz.

15. A method comprising:
energizing a first magnetic beacon with a first offset frequency concurrently with a second magnetic beacon with a second offset frequency;
receiving, at one or both of the first magnetic beacon and the second magnetic beacon, magnetic field measurement data measured by an ingestible device; and
generating, by a controller, location data of the ingestible device based on the magnetic field measurement data.

16. The method of claim 9, wherein the ingestible device comprises a receiving coil and a transmission coil, wherein the transmission coil uses a frequency greater than 50 MHz.

17. The method of claim 9, wherein the ingestible device comprises a chip including a radio frequency low noise amplifier (RFLNA) configured to amplify measurements of the magnetic fields generated by the first magnetic beacon and the second magnetic beacon.

18. The method of claim 9, wherein generating the location data comprises applying a neural network on the measurement data.

19. The method of claim 9, wherein energizing the first magnetic beacon and the second magnetic beacon comprises energizing the first magnetic beacon and the second magnetic beacon with a sustained current.

20. The method of claim 9, wherein the first magnetic beacon and the second magnetic beacon utilize a carrier frequency in a range of about 1-100 MHz.
